SEQUENCE LISTING (33 sequences)

SEQ ID NO: 1 - protein, 108 aa, Ginkgo biloba
  DOMAIN (1)..(108): Oligomerization domain of LEAFY protein
MARKELSSLEELFRHYGVRYMTLTKMVEMGFTVNTLVNMTEQELDDVIRTLVDIYRVDLLVGEKYGIKSAVRAEKRRLDELERKKLDLFVDVDGKRKADENALDTLSQ

SEQ ID NO: 2 - protein, 8 aa, Artificial Sequence
  Strep tag
WSHPQFEK

SEQ ID NO: 3 - protein, 26 aa, Artificial Sequence
  Histidine Tag
MKHHHHHHPMSDYDIPTTENLYFQGA

SEQ ID NO: 4 - protein, 21 aa, Artificial Sequence
  Histidine Tag
MKHHHHHHPMSDYDIPTTEGA

SEQ ID NO: 5 - protein, 16 aa, Artificial Sequence
  Histidine Tag
MKHHHHHHPMSDYDGA

SEQ ID NO: 6 - protein, 11 aa, Artificial Sequence
  Histidine Tag
MKHHHHHHPGA

SEQ ID NO: 7 - protein, 9 aa, Artificial Sequence
  Histidine Tag
MHHHHHHGA

SEQ ID NO: 8 - protein, 9 aa, Artificial Sequence
  Poly-Lysine Tag
MKKKKKKGA

SEQ ID NO: 9 - protein, 9 aa, Artificial Sequence
  Poly-Glutamate Tag
MEEEEEEGA

SEQ ID NO: 10 - protein, 9 aa, Artificial Sequence
  Poly-Glutamate/Lysine Tag
MEEEKKKGA

SEQ ID NO: 11 - protein, 9 aa, Artificial Sequence
  Poly-Proline Tag
MPPPPPPGA

SEQ ID NO: 12 - protein, 25 aa, Artificial Sequence
  Part configured to line the honeycomb cells
KKLDLFVDVDGKRKADENALDTLSQ

SEQ ID NO: 13 - protein, 25 aa, Artificial Sequence
  Part configured to line the honeycomb cells
CKLDLFVDVDGKRKADENALDTLSQ

SEQ ID NO: 14 - protein, 6 aa, Artificial Sequence
  Spacer
GGSGGS

SEQ ID NO: 15 - protein, 134 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKLDLFVDVDGKRKADENALDTLSQ

SEQ ID NO: 16 - protein, 134 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERCKLDLFVDVDGKRKADENALDTLSQ

SEQ ID NO: 17 - protein, 114 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKLDA

SEQ ID NO: 18 - protein, 86 aa, Artificial Sequence
  Protein of the invention
MHHHHHHGAMARKELSSLEELFRHYGVRYMTLTKMVEMGFTVNTLVNMTEQELDDVIRTLVDIYRVDLLVGEKYGIKSAVRAEKRR

SEQ ID NO: 19 - protein, 118 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGGSGGSC

SEQ ID NO: 20 - protein, 120 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGGSGGSECG

SEQ ID NO: 21 - protein, 123 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGGSGGSHHHHHH

SEQ ID NO: 22 - protein, 123 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGGSGGSCHCHCH

SEQ ID NO: 23 - protein, 124 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGGSKKKKKKGGSC

SEQ ID NO: 24 - protein, 113 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGC

SEQ ID NO: 25 - protein, 119 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGCHCHCHC

SEQ ID NO: 26 - protein, 98 aa, Artificial Sequence
  Protein of the invention
MHHHHHHGAMARKELSSLEELFRHYGVRYMTLTKMVEMGFTVNTLVNMTEQELDDVIRTLVDIYRVDLLVGEKYGIKSAVRAEKRRLDELERKKGGSC

SEQ ID NO: 27 - protein, 103 aa, Artificial Sequence
  Protein of the invention
MHHHHHHGAMARKELSSLEELFRHYGVRYMTLTKMVEMGFTVNTLVNMTEQELDDVIRTLVDIYRVDLLVGEKYGIKSAVRAEKRRLDELERKKGGSHHHHHH

SEQ ID NO: 28 - protein, 126 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGGSTGTSVLIATPGV

SEQ ID NO: 29 - protein, 111 aa, Artificial Sequence
  Protein of the invention
MKHHHHHHPGAMARKELSSLEELFRHYGVRYMTLTKMVEMGFTVNTLVNMTEQELDDVIRTLVDIYRVDLLVGEKYGIKSAVRAEKRRLDELERKKGGSTGTSVLIATPGV

SEQ ID NO: 30 - protein, 126 aa, Artificial Sequence
  Protein of the invention
MKHHHHHHPMSDYDIPTTENLYFQGAMARKELSSLEELFRHYGVRYMTLTKMVEMGFTVNTLVNMTEQELDDVIRTLVDIYRVDLLVGEKYGIKSAVRAEKRRLDELERKKGGSWAGAKRLVLRRE

SEQ ID NO: 31 - protein, 111 aa, Artificial Sequence
  Protein of the invention
MKHHHHHHPGAMARKELSSLEELFRHYGVRYMTLTKMVEMGFTVNTLVNMTEQELDDVIRTLVDIYRVDLLVGEKYGIKSAVRAEKRRLDELERKKGGSWAGAKRLVLRRE

SEQ ID NO: 32 - protein, 15 aa, Artificial Sequence
  Peptide capable of interacting and reducing metal salts
GGSTGTSVLIATPGV

SEQ ID NO: 33 - protein, 15 aa, Artificial Sequence
  Peptide capable of interacting and reducing metal salts
GGSWAGAKRLVLRRE

(19) United States
(12) Patent Application Publication
(10) Pub. No.: US 2022/0380486 A1
(43) Pub. Date: Dec. 1, 2022

(54) METHOD FOR SELF-ASSEMBLY OF A PROTEIN ON A SUBSTRATE IN A THREE-DIMENSIONAL HONEYCOMB STRUCTURE

(71) Applicants: COMMISSARIAT A L'ENERGIE ATOMIQUE ET AUX ENERGIES ALTERATIVES, Paris (FR); CENTRE NATIONAL DE LA RECHERCHE SCIENTIFIQUE, Paris Cedex 16 (FR); UNIVERSITÉ GRENOBLE ALPES, Saint-Martin-D'Heres (FR)

(72) Inventors: Pierre-Henri ELCHINGER, La Buisse (FR); Renaud Dumas, Bourgoin Jallieu (FR); Elise Jacquier, Grenoble (FR); Pierre-Henri Jouneau, Grenoble (FR); François Parcy, Grenoble (FR); Raluca Tiron, Grenoble (FR)

(21) Appl. No.: 17/627,506

(22) PCT Filed: Jun. 2, 2020
(86) PCT No.: PCT/FR2020/050932

§ 371 Date: Jan. 14, 2022

(30) Foreign Application Priority Data
Jul. 18, 2019 (FR) FR1908150

(51) Int. Cl. C07K 17/14; C07K 1/30

(52) U.S. Cl. CPC C07K 17/14; C07K 1/306

(57) ABSTRACT

A method for self-assembly of a protein in a three-dimensional honeycomb structure, comprising the following consecutive steps: providing a solution comprising a solvent and a protein, the protein comprising a sequence of amino acids corresponding to an oligomerisation domain of a LEAFY protein, for example to the oligomerisation domain of Ginkgo biloba, in fusion with a tag, placing the solution in contact with a substrate, evaporating the solvent in order to crystallise the protein, the oligomerisation domain crystallising in the form of a primary helix, each primary helix interacting with six other primary helixes, whereby a three-dimensional honeycomb protein structure is obtained perpendicular to the substrate, the protein structure being attached to the substrate by the tag.

### TECHNICAL FIELD

The present invention is concerned with the general field of protein crystallisation.

The invention relates to a method for self-assembling proteins on a substrate into a three-dimensional honeycomb structure.

The invention is particularly interesting since it makes it possible to obtain a stable nanostructured material, the patterns of which are very regular.

The invention also relates to the thus obtained assembly, formed by the substrate and the protein structure. Such an assembly forms a grafting platform for a wide variety of molecules.

The invention also relates to a use of such an assembly. The invention finds applications in many industrial fields, especially in the fields of photolithography, catalysis, optics, or even for membrane manufacture.

The invention also relates to a method for manufacturing nanopillars from such an assembly.

### STATE OF PRIOR ART

Currently, to generate protein self-assembly on a surface, two approaches are used: the so-called “top-down” approach and “bottom-up” approach.

The top-down type approach consists in locally modifying the surface of a substrate in order to create binding sites that can interact with proteins. The self-assembly of proteins is then conditioned by their attachment to these binding sites. However, such an approach requires structuring the substrate at the nano- or micrometre scale, which requires additional steps and makes the method more time-consuming and expensive.

The “bottom-up” type approach relies on intrinsic properties of proteins to interact with each other. Several types of “bottom-up” methods have been reported. These include, especially:
inverse emulsion techniques, based on the evaporation of a protein microemulsion on a surface (“inverse emulsion breath-figure”),
Langmuir-Blodgett techniques, relying on the production of a pressure-induced monolayer protein film on the surface of a liquid, followed by the deposition of this film onto a solid surface, and
2D recrystallisation methods for “S-layer” proteins.

For example, in Gonen et al (“Design of ordered two-dimensional arrays mediated by noncovalent protein-protein interfaces”, Science (2015), 348, 6241, 1365-1368), the computer-aided design of protein interfaces allowed the definition of different 2D array architectures. Protein crystals up to 1 μm long and 8 nm thick are obtained.

In the paper of Sayou et al (“A SAM oligomerisation domain shapes the genomic binding landscape of the LEAFY transcription factor”, Nature Communications (2016), 7, 11222), the crystal structure of the oligomerisation domain of Ginkgo biloba has been studied in solution. The authors have shown that the head-tail oligomerisation of the monomers leads to the formation of a helix.

The different architectures obtained are 2D architectures, formed by single or double layers. However, these protein architectures do not have a large stability.

### DISCLOSURE OF THE INVENTION

One purpose of the present invention is therefore to provide a method for making a protein self-assembly that is stable over time and/or solvent-resistant.

For this, the present invention provides a method for self-assembling a protein into a three-dimensional honeycomb structure comprising the following successive steps of:
providing a solution comprising a solvent and a protein, the protein comprising an amino acid sequence corresponding to an oligomerisation domain of a LEAFY protein, for example the oligomerisation domain of Ginkgo biloba, fused to a tag,
contacting the solution with a substrate,
evaporating the solvent to crystallise the protein, for example by vapour diffusion, the oligomerisation domain crystallising as a primary helix, each primary helix interacting with six other primary helices, whereby a three-dimensional honeycomb protein structure is obtained, perpendicular to the substrate, the protein structure being bound to the substrate by the tag.

The invention differs fundamentally from prior art in that a tag is used which allows growth perpendicular to the substrate surface (i.e. growth upwards and not along the substrate) and thus a three dimensional architecture is obtained. The primary helices form regularly spaced cells, which can advantageously be functionalised, within the protein structure. Such an architecture has very good stability. Such a method is relatively simple to implement since the protein structure self-assembles spontaneously upon evaporating the solvent.

Advantageously, the method includes an additional step in which another protein corresponding to the oligomerisation domain of the tag-free LEAFY protein is added to the already formed honeycomb structure, whereby the height of the honeycomb structure is increased perpendicular to the substrate.

Advantageously, the substrate is made of a material selected from a metal, a metalloid or a carbonaceous material. The choice of the substrate material and especially its hydrophilic/hydrophobic properties affects the substrate/tag affinity. This will influence the surface area of the self-assembled structure and the coverage rate of the structure with the substrate.

The invention also relates to an assembly obtained by such a method, the assembly comprising a substrate covered with a protein self-assembly in a three-dimensional honeycomb structure,

the protein comprising an amino acid sequence corresponding to an oligomerisation domain of a LEAFY protein, for example the oligomerisation domain of Ginkgo biloba, fused to a tag,

the oligomerisation domain being crystallised as a primary helix, each primary helix interacting with six other primary helices, the structure being bound perpendicular to the substrate through the tag.

Advantageously, the pitch between each primary helix is less than 10 nm. By pitch, it is meant the distance between the centre of the lumen of two adjacent primary helices.

Advantageously, the internal diameter of a primary helix ranges from 4 nm to 6 nm, for example 5 nm.

Advantageously, the self-assembly covers an area of at least 50 μm².

Advantageously, the substrate is porous. This embodiment is particularly advantageous, especially for making membranes whose pore size is defined by the honeycomb structure cells.

According to a first advantageous alternative, the protein is functionalised with a metal (metal nanoparticles), a metal salt, an inorganic complex or an organic molecule. This embodiment is particularly advantageous for making metal nanopillars, nanocatalysts or nanophosphors.

The organic molecule may be, for example, a chromophore or a fluorophore. According to one particularly advantageous embodiment, the organic molecule is a peptide that can interact with metals (such as gold for example) and/or can reduce metal salts to make metal nanopillars.

According to another advantageous alternative, the protein is functionalised with quantum boxes (also called quantum dots QDs).

According to another advantageous alternative, the honeycomb structure is metallised, for example with gold. This embodiment is particularly advantageous for forming regularly spaced metal rings with nanometre dimensions, for optical applications.

According to one particularly advantageous embodiment, the C-terminal sequence of the protein is modified with a peptide sequence that can interact with metals (such as gold, for example) and/or can reduce metal salts, for example, to make metal nanopillars.

Such a structure has many advantages:
the regularity of the patterns of the structure deposited is very high, even higher than existing technologies (block copolymers especially),
it is possible to functionalise the interior of the wells with organic and/or inorganic molecules,
the structure can be deposited onto many planar or curved substrates,
it is possible to control the diameter of the honeycomb structure cells as well as the pitch between cells, using different protein architectures,
the protein structure bound to the substrate is very stable,
there is no need to structure the substrate beforehand in order to bind the protein structure.

The invention also relates to a use of an assembly as defined above, to develop applications in nanotechnology, for example as a photolithography mask, as a signal amplifier of an optical sensor, as a membrane, or as a catalyst.

The invention also relates to a method for manufacturing metal nanopillars comprising the following successive steps of:
providing an assembly comprising a substrate covered with a protein self-assembly in a three-dimensional honeycomb structure, the protein comprising an amino acid sequence corresponding to an oligomerisation domain of a LEAFY protein, for example the oligomerisation domain of Ginkgo biloba, fused to a tag, and specific amino acids, capable of being functionalised by a metal or metal salt, lining the honeycomb cells, the oligomerisation domain being crystallised as a primary helix, each primary helix interacting with six other primary helices, the structure being bound perpendicular to the substrate by the tag,
grafting, onto the specific amino acids, a metal (for example in the form of metal nanoparticles), an inorganic complex or a metal salt and reducing it, to form metal nanopillars in the honeycomb cells.

Further characteristics and advantages of the invention will become apparent from the following further description.

No need to say this further description is given only by way of illustration of the object of the invention and should in no way be construed as a limitation of that object.

### BRIEF DESCRIPTION OF THE DRAWINGS

The present invention will be better understood upon reading the description of exemplary embodiments given purely by way of illustrating and in no way limiting purposes, with reference to the appended drawings in which:

FIG. 1A schematically represents, in different views, the structure of two monomers of the oligomerisation domain of an interacting LEAFY protein (PDB code 4UDE), according to one particular embodiment of the invention; for better legibility, the histidine tag and the disordered C-terminal part of the monomers are not represented.

FIG. 1B schematically represents, in different views, the structure of the helix formed by the head-tail oligomerisation of the monomers of the oligomerisation domain of a LEAFY protein, according to one particular embodiment of the invention.

FIG. 1C schematically represents, in different views, details of the interaction of two primary helices formed by oligomerisation of the oligomerisation domain of a LEAFY protein, according to one particular embodiment of the invention; for better legibility, only the monomers of one helix are differentiated.

FIG. 1D schematically represents a primary helix interacting with 6 other primary helices to form a honeycomb protein structure, according to one particular embodiment of the invention.

FIG. 2 is an electron microscopy (STEM) picture of a self-assembly of the oligomerisation domain of a histidine tag-free LEAFY protein into a honeycomb structure, in parallel to the surface of a carbon substrate; the inset schematically represents the orientation of a primary helix in the structure.

FIGS. 3A and 3B are electron microscopy (STEM) pictures of a self-assembly of a protein comprising an oligomerisation domain of a histidine-tagged LEAFY protein, in a honeycomb structure, perpendicular to the surface of a carbon substrate, according to one particular embodiment of the invention and at different scales; the inset of FIG. 3B schematically represents the orientation of a primary helix in the structure.

FIGS. 4A and 4B are electron microscopy (STEM) pictures of a protein self-assembly on a carbon substrate, at different scales, according to one particular embodiment of the invention.

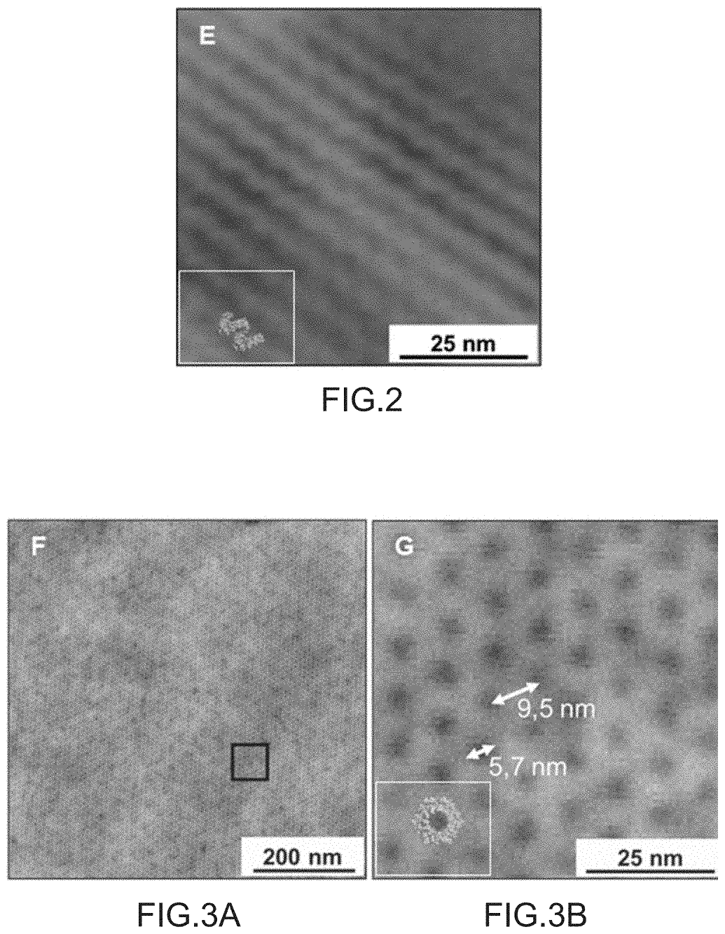

FIGS. 2, 3A, 3B, 4A and 4B were obtained by black-tagging, with uranyl acetate, the amino acids lining the lumen of the primary helix wells.

The different parts represented in the figures are not necessarily represented on a uniform scale, to make the figures more legible.

The various possibilities (alternatives and embodiments) are to be understood as not being exclusive of each other and may be combined with one another.

Further, in the following description, terms that depend on the orientation, such as “on”, “over”, “below”, “under”, etc. of a structure are applied assuming that the structure is oriented as illustrated in the figures.

### DETAILED DISCLOSURE OF PARTICULAR EMBODIMENTS

Here and hereafter, the self-assembly method is described for a protein. However, the invention is applicable to peptides, polypeptides and more generally to amino acid sequences homologous to the amino acid sequence of the oligomerisation domain of the LEAFY protein.

The self-assembly method for obtaining a three-dimensional honeycomb protein structure comprises the following successive steps of:

a) providing a solution containing a protein of interest,

b) contacting the solution with the surface of a substrate,

c) crystallising the protein, whereby a three-dimensional self-assembly of the protein into a honeycomb structure is obtained, perpendicular to the surface of the substrate.

In step a), the protein of interest used to form the three-dimensional honeycomb protein structure on the substrate has a primary amino acid sequence comprising:
a tag,
an oligomerisation domain of a LEAFY protein,
a portion for lining the honeycomb cells.

By oligomerisation domain, it is intended an amino acid sequence that allows proteins to assemble in sequence into small chains.

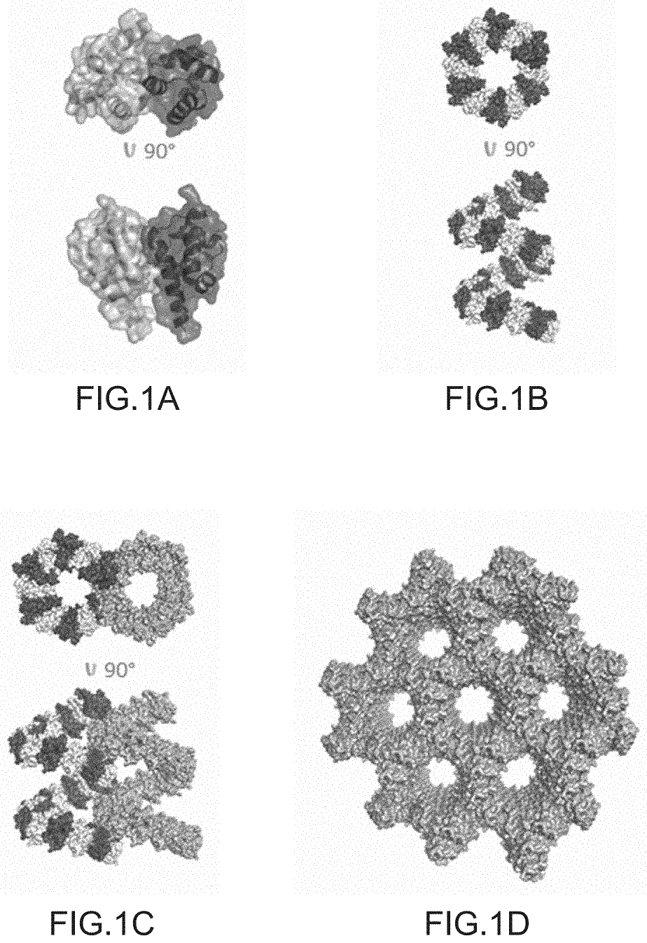

The oligomerisation domain is that of the LEAFY protein. The LEAFY transcription factor (also known as LFY) is involved in developmental processes in plants, especially flower formation. The oligomerisation of the LEAFY protein is a head-tail oligomerisation (FIGS. 1A, 1B, 1C). Such oligomerisation allows, in contrast to head-tail interactions, the self-assembly of monomers in the form of a primary helix. The primary helix of the oligomerisation domain of the protein forms a substantially helical coil, the internal diameter of which forms a cell (also called a well or lumen). The walls of the cell are lined with the C-terminal end of each monomer. The groove of one primary helix interacts in parallel with the groove of 6 other primary helices to generate a honeycomb organisation (FIG. 1D). In other words, one primary helix is interlocked with six other primary helices in a honeycomb (or hexagonal) structure. Bonds between the primary helices are hydrogen and ionic bonds.

By way of illustrating and not limiting purposes, the oligomerisation domain can be that of Arabidopsis thaliana (AtLFY), Ginkgo biloba (GbLFY), Ceratopteris richardii (CrLFY) or Physcomitrella patens (PpLFY).

For example, the oligomerisation domain of the Ginkgo biloba LEAFY protein is (here and hereafter all amino acid sequences are noted from N-terminal to C-terminal):

MARKELSSLEELFRHYGVRYMTLTKMVEMGFTVNTLVNMTEQELDDVIR
TLVDIYRVDLLVGEKYGIKSAVRAEKRRLDELERKKLDLFVDVDGKRKA
DENALDTLSQ (SEQ ID NO: 1 in the appended sequence
listing).

The orientation of the self-assembly is determined by the addition of a tag. Generally, the tag is a short amino acid sequence (typically having 6 to 30 amino acids).

In the absence of this tag, self-assembly grows parallel to the surface, that is in the plane of the substrate surface in contact with the protein of interest solution in step b) (FIG. 2). In the presence of this tag, the assembly grows perpendicular to the substrate surface, that is perpendicular to the plane of the substrate surface in contact with the protein of interest solution in step b). FIGS. 3A and 3B represent a self-assembly obtained with a tag (histidine tag): the growth is perpendicular to the substrate surface. The protein tag allows the protein structure to be bound to the substrate surface.

Several types of tags can be used, such as
tags comprised of positively charged amino acids (for example from 6 to 30 and preferably from 6 to 10 residues among lysine (K) or arginine (R)) allowing to bind to a negatively charged surface; for example, such tags could be used with silicon oxide surfaces,
tags comprised of negatively charged amino acids (for example from 6 to 30 and preferably from 6 to 10 residues among glutamate (E) or aspartate (D)) allowing to bind to a positively charged surface; for example such tags can be used with surfaces treated with amylamine,
tags comprised of hydrophobic amino acids (for example from 6 to 30 and preferably from 6 to 10 residues among leucine (L), valine (V), isoleucine (I), methionine (M), phenylalanine (F), tryptophan (W), proline (P)) allowing to bind to a hydrophobic surface; for example, such tags may be used with surfaces treated with silanes,
tags comprised of polar amino acids (for example from 6 to 30 and preferably from 6 to 10 residues among serine (S), threonine (T), tyrosine (Y)) allowing to bind to a polar surface; for example, such tags may be used with silicon, aluminium or titanium oxide surfaces,
tags comprised of amino acids capable of binding to metals (for example from 6 to 30 and preferably from 6 to 10 residues among cysteine (C), histidine (H), glutamate (E), aspartate (D)) allowing to bind to metal surfaces; for example, such tags may be used with surfaces of palladium, platinum, molybdenum, cobalt or gold,
tags comprised of specific amino acids allowing a high affinity interaction of the receptor/ligand type on functionalised surfaces, such as a Strep tag (WSHPQFEK; SEQ ID NO: 2 in the appended sequence listing) on a surface functionalised with streptavidin.

The tag starts with an initiator methionine (M).

The tag may end with a sequence of, for example, 2-20 residues acting as a linker, for example GA.

According to one particularly advantageous embodiment, the tag is a histidine tag (also called a polyhistidine tag or histidine tag). The histidine tag comprises at least six histidine amino acids. It may include more than six histidine amino acids. It may also comprise other amino acids. By way of illustrating and not limiting purposes, one of the following sequences may be chosen:

-MKHHHHHHPMSDYDIPTTENLYFQGA (SEQ ID NO: 3 in the appended
sequence listing),
-MKHHHHHHPMSDYDIPTTEGA (SEQ ID NO: 4 in the appended sequence
listing)
-MKHHHHHHPMSDYDGA (SEQ ID NO: 5 in the appended sequence
listing)
-MKHHHHHHPGA (SEQ ID NO: 6 in the appended sequence listing)
-MHHHHHHGA (SEQ ID NO: 7 in the appended sequence listing)

All these sequences have been tested and allow anchoring of the honeycomb structure perpendicular to the substrate surface.

Other constructs including a poly-Lysine, poly-Glutamate, poly-Glutamate/Lysine and poly-Proline tag have also been obtained:
Poly-Lysine sequence: MKKKKKKGA (SEQ ID NO: 8 in the appended sequence listing),
Poly-Glutamate sequence: MEEEEEEGA (SEQ ID NO: 9 in the appended sequence listing),
Poly-Glutamate/Lysine sequence: MEEEKKKGA (SEQ ID NO: 10 in the appended sequence listing),
Poly-Proline sequence: MPPPPPPGA (SEQ ID NO: 11 in the appended sequence listing).

Hereafter, a histidine tag will be described. Any other tag, especially one with equivalent size, which non-specifically or specifically links to a substrate, and allows the protein structure to grow perpendicular to the substrate, may be used.

The amino acid sequence also includes a portion for lining the cells of the honeycomb. For example, this portion corresponds to the sequence:

KKLDLFVDVDGKRKADENALDTLSQ (SEQ ID NO: 12 in the
appended sequence listing)

This part is fully adjustable. It can be removed. It can also be modified by deletion, substitution, or insertion. Modifications to this part do not affect the self-assembly ability of the protein. Advantageously, it is modified by substitution or insertion so as to introduce one or more specific amino acids capable of being functionalised by a metal ion, a metal or an organic molecule. For example, a cysteine (also called cysteine (C) residue) is chosen as the specific amino acid, for specifically grafting molecules carrying a maleimide or iodoacetamide group or metal salts.

The sequence of the portion lining the cells modified by substitution can be for example: CKLDLFVDVDGKRKADENALDTLSQ (SEQ ID NO: 13 in the appended sequence listing).

By way of illustrating and not limiting purposes, the following table lists different amino acid sequences of the protein that can be used: a primary sequence and mutant sequences. The mutant sequences are obtained by modifying the tag and/or the C-terminal sequence lining the cells of the primary sequence. These mutants have been tested and self-assemble into honeycombs perpendicular to the surface. In the examples given, the C-terminal can be shortened or replaced by combinations of amino acids (Cysteine, Histidine, Lysine; Glutamate, Glycine) capable of interacting with molecules and preceded by a shorter or longer spacer. The spacer can be comprised of the amino acids Glycine and Serine. For example, the spacer can be GGSGGS (SEQ ID NO: 14 in the appended sequence listing), GGS and G.

| | |
|---|---|
| Primary amino acid sequence of the protein | MKHHHHHHPMSDYDIPTTENLYFQGAMARK |
| (SEQ ID NO: 15 in the appended sequence | ELSSLEELFRHYGVRYMTLTKMVEMGFTVNTLV |
| listing) | NMTEQELDDVIRTLVDIYRVDLLVGEKYGIKSAV |
| | RAEKRRLDELERKKLDLFVDVDGKRKADENALD |
| | TLSQ |
| Sequence of a mutant with the previously | MKHHHHHHPMSDYDIPTTENLYFQGAMARK |
| described substitution (SEQ ID NO: 16 in the | ELSSLEELFRHYGVRYMTLTKMVEMGFTVNTLV |
| appended sequence listing) | NMTEQELDDVIRTLVDIYRVDLLVGEKYGIKSAV |
| | RAEKRRLDELERCKLDLFVDVDGKRKADENALD |
| | TLSQ |
| Sequence of a mutant of the C-terminal | MKHHHHHHPMSDYDIPTTENLYFQGAMARK |
| part (SEQ ID NO: 17 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been partially removed and modified | RAEKRRLDELERKKLDA |
| Sequence of a mutant of the C-terminal | MHHHHHHGAMARKELSSLEELFRHYGVRYMT |
| part and the histidine tag (SEQ ID NO: 18 in | LTKMVEMGFTVNTLVNMTEQELDDVIRTLVDIY |
| the appended sequence listing): the C- | RVDLLVGEKYGIKSAVRAEKRR |
| terminal part has been removed and the | |
| histidine tag has been modified | |
| Sequence of a mutant of the C-terminal | MKHHHHHHPMSDYDIPTTENLYFQGAMARK |
| part (SEQ ID NO: 19 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been modified | RAEKRRLDELERKKGGSGGSC |
| Sequence of a mutant of the C-terminal | MKHHHHHHPMSDYDIPTTENLYFQGAMARK |
| part (SEQ ID NO: 20 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been modified | RAEKRRLDELERKKGGSGGSECG |
| Sequence of a mutant of the C-terminal | MKHHHHHHPMSDYDIPTTENLYFQGAMARK |
| part (SEQ ID NO: 21 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been modified | RAEKRRLDELERKKGGSGGSHHHHHH |
| Sequence of a mutant of the C-terminal | MKHHHHHHPMSDYDIPTTENLYFQGAMARK |
| part (SEQ ID NO: 22 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been modified | RAEKRRLDELERKKGGSGGSCHCHCH |
| Sequence of a mutant of the C-terminal | MKHHHHHHPMSDYDIPTTENLYFQGAMARK |
| part (SEQ ID NO: 23 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been modified | RAEKRRLDELERKKGGSKKKKKKGGSC |
| Sequence of a mutant of the C-terminal | MKHHHHHHPMSDYDIPTTENLYFQGAMARK |
| part (SEQ ID NO: 24 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been modified | RAEKRRLDELERKKGC |
| Sequence of a mutant of the C-terminal | MKHHHHHHPMSDYDIPTTENLYFQGAMARK |
| part (SEQ ID NO: 25 in the appended | ELSSLEELFRHYGVRYMTLTKMVEMGFTVNTLV |
| sequence listing): the C-terminal part has | NMTEQELDDVIRTLVDIYRVDLLVGEKYGIKSAV |
| been modified | RAEKRRLDELERKKGCHCHCHC |
| Sequence of a mutant of the C-terminal | MHHHHHHGAMARKELSSLEELFRHYGVRYMT |
| part and histidine tag (SEQ ID NO: 26 in the | LTKMVEMGFTVNTLVNMTEQELDDVIRTLVDIY |
| appended sequence listing): the C-terminal | RVDLLV |
| and histidine tag have been modified | GEKYGIKSAVRAEKRRLDELERKKGGSC |
| Sequence of a mutant of the C-terminal | MHHHHHHGAMARKELSSLEELFRHYGVRYMT |
| part and histidine tag (SEQ ID NO: 27 in the | LTKMVEMGFTVNTLVNMTEQELDDVIRTLVDIY |
| appended sequence listing): the C-terminal | RVDLLVGEKYGIKSAVRAEKRRLDELERKKGGSH |
| and histidine tag have been modified | HHHHH |

According to a first alternative, the protein of interest can be synthesised by synthetic biology.

According to a second preferred alternative, the protein of interest is produced by a bacterium. For this, the protein of interest can be expressed in a commercial strain, advantageously by a strain of E. coli bacteria. The protein of interest is then purified from the soluble extract of the bacteria that produced it. The purification consists, for example, in collecting the bacteria by centrifugation, breaking their membrane by sonication and then separating the soluble proteins containing the protein of interest by centrifugation. The protein of interest is then purified one or more times.

This can be an affinity purification on a Nickel-containing resin (for example Nickel-Sepharose type) and/or on a permeation gel (for example Superdex 200 type, marketed by GE Healthcare). Only proteins with the histidine tag are retained on the nickel-containing resin.

The protein is soluble in an aqueous buffer, preferably Tris-HCl (pH between 7.0 and 9.0 at a concentration between 10 and 100 mM) containing a thiol function reducer (DTT (Dithiothreitol) or TCEP (Tris(2-carboxyethyl)phosphine hydrochloride) at a concentration of 1 mM) to form a protein solution.

The protein solution is advantageously mixed with a crystallisation solution (consisting of a buffer and a salt). This may be a 50/50 volume mixture. The crystallisation solution comprises, for example, Tris-HCl (the buffer) and ammonium sulphate (the salt). More generally, a so-called Good buffer (for example ADA, HEPES, CAPS) may also be used at concentrations between 25 and 200 mM and a pH between 6.5 and 8.5. Other salts such as lithium sulphate may also be used.

The concentration of the protein in the solution used in step a) can range from 0.5 mg/mL to 5 mg/mL.

In step b), a volume V₁ of the solution containing the protein of interest is contacted with the substrate. The substrate can be of different kinds. For example, it may be metallic, made of a metalloid element or of a carbonaceous material. By way of illustration, it may be silicon or carbon. For example, a silicon wafer or a silicon or carbon microscopy grid may be chosen. The substrate may be transparent. By transparent, it is meant that the substrate has a transmittance greater than 50% in the visible range, i.e. from 350 nm to 750 nm, and preferably greater than 70% in the visible range.

Advantageously, the substrate is hydrophilic in order to favour its covering by the solution containing the protein of interest and/or the interactions with the histidine tag.

The surface area of the substrate to be functionalised may be greater than 50 μm², or even greater than 1 mm². It can be, for example, about 7 mm².

A drop of solution containing the protein of interest, or a larger volume of such solution, may be deposited onto the surface to be treated or the surface to be treated may be deposited onto the solution containing the protein of interest.

In step b), the protein solution may advantageously locally cover the substrate. According to one particularly advantageous alternative embodiment, a step during which a so-called protective layer is formed locally on the substrate, for example during a photolithography step through a mask, can be carried out prior to step b). The substrate then comprises parts covered with the protective layer and parts not covered with the protective layer. This makes it possible to delimit zones on the substrate to be functionalised with the protein structure. The protein solution is in contact with the surface of the substrate at parts not covered with the protective layer. Walls of the protective layer act as a guide upon growing the protein structure.

In step c), the protein is crystallised to form the protein structure. Crystallisation consists in making the protein change from a soluble state to a solid, ordered state. The solution containing the protein is gradually evaporated to increase concentration of the protein until it crystallises.

Self-assembly of the protein on the surface of the substrate is advantageously carried out in a closed chamber, for example in a crystallisation chamber.

The enclosure contains, advantageously, a crystallisation tank with a volume V₂ containing the crystallisation buffer solution. A volume V₂ greater than the volume V₁ will be chosen. The volume V₂ ranges, for example, from 500 μL to 5 mL. Once the enclosure is sealed, vapour diffusion from the drop containing the protein solution to the reservoir is initiated and results in slow evaporation of the solvent from the protein solution allowing the protein to self-assemble on the surface.

The crystallisation is advantageously carried out at room temperature (i.e. 20 to 25° C.) and at room pressure (in the order of 1 bar). The duration of step c) ranges, advantageously, from 4 h to 48 h. This duration depends on the surface area of the substrate to be functionalised as well as the solution volume. The self-assembly is visible from 4 hours and reaches an optimum after 24 hours.

At the end of the method, an assembly is obtained comprising a substrate covered with the crystallised and self-assembled protein in a three-dimensional honeycomb structure perpendicular to the surface of the substrate. It has a controlled height corresponding to the stack of the different monomers. The height of the protein structure averages 18 nm with the protein containing the histidine tag but may be higher using a second growth step in the presence of the protein without the histidine tag. The internal diameter of the honeycomb cells ranges, for example, from 4 nm to 6 nm. The pitch between primary helices is less than 10 nm. It ranges, for example, from 8 nm to 10 nm.

By way of illustration, the protein architecture obtained with the oligomerisation domain of the Ginkgo biloba LEAFY protein has the following characteristics (FIGS. 3A and 3B):
one turn of the primary helix corresponds to 12 monomers,
the height of the three-dimensional structure is about 31 nm, corresponding to the stack of 40 monomers,
the pitch between the centre of the lumens of two adjacent primary helices is 9.5 nm,
the width of the groove within a single primary helix ranges from 4 to 5 nm,
the internal diameter of the primary helix is 5 nm.

These characteristics can be measured by atomic force microscopy (AFM) or transmission electron microscopy (STEM), for example STEM, TEM after a standard protein structure staining step using uranyl acetate.

Parameters of the self-assembled protein structure, such as well diameter and well height can be easily modified.

For example, by increasing the number of amino acids lining the well wall, the well diameter can be reduced. Varying the number of amino acids lining the interior of the wells also allows the number of molecules grafted into the cells of the protein structure to be modulated.

The height of the self-assembled structure can be increased by the addition of the oligomerisation domain, which lacks the histidine tag, in other words by adding monomers of the oligomerisation domain to the self-assembly so as to grow the primary helices of the protein structure. This modification makes it possible, for example, to increase the number of grafted (inorganic, organic or mixed) molecules, to have a nanolithography mask with a greater thickness or even to increase the height of the pillars that can grow within the self-assembly.

The surface area of the self-assembled structure and the coverage rate of a substrate can be increased, for example, by varying affinity of the substrate for the histidine tag.

It is also possible to modify the shape, diameter and spacing of the wells by choosing different protein architectures. The identification of these protein architectures or protein domains capable of making head-tail interactions can be done in the Protein Data Bank (PDB) collecting all known protein structures.

The resulting three-dimensional self-assembled honeycomb structure is a biomaterial that can be used for many applications.

According to one particular embodiment, the structure can be used as a membrane, when the self-assembled structure is formed on a porous surface, the permeability threshold is determined by the internal diameter of the primary helices.

According to another embodiment, the self-assembled structure can be covered, totally or partially, by a metallic layer, for example of gold. It may be metallised by physical vapour deposition, chemical vapour deposition, chemical deposition or electrochemical deposition. Such a structure can be used to influence behaviour of a light wave at the surface of a substrate. This is of particular interest for amplifying the signal of an optical sensor, such as a sensor selected from among sensors based on surface plasmon resonance (SPR), surface-enhanced Raman scattering (SERS) sensors, or for surface-enhanced infrared spectroscopy (SEIRAS). It is, for example, possible to form regularly spaced gold rings by covering the upper part of the structure with gold.

According to another embodiment, the self-assembled structure can be used as a photolithography mask: as the self-assembly generates wells of regular diameter and spacing (for example 5 nm in diameter and 9 nm apart), it can be used as a photolithography mask to etch a support through the honeycomb cells. Etching could be, for example, of the chemical (HF) type on silicon support or with ultraviolet (UV) on a photosensitive surface.

According to another embodiment, the self-assembly can serve as a platform/support for grafting inorganic (metal ions, inorganic complexes, QDs or metal particles, for example) and organic (fluorophores or peptides, for example) molecules. For this, a protein with a sequence comprising one or more amino acids that can be functionalised by metal salts that can be reduced to metal nanoparticles, by inorganic complexes with some catalytic activity, by organic molecules or by QDs or nanoparticles will be chosen. Alternatively, the C-terminal part of the protein can be replaced by an amino acid sequence that allows both the attraction of metal salts and their reduction in situ without needing to add a reducer.

By way of illustration, on the basis of a self-assembly of 31 nm in height alone (that is a stack of 40 monomers per well) and approximately 12,000 wells per μm², it is estimated that approximately 480,000 molecules/μm² (that is 48,000 billion/cm²) are bound. Such a self-assembly has a high surface area and therefore allows a high quantity of molecules to be bound. The position and specificity of grafting can be controlled by the amino acid composition lining the lumen of the wells and the reactivity of the compounds to be grafted.

By way of example, functionalisation operations can be performed on naturally occurring or modified amino acids of the C-terminal end of the protein that are found in the lumen of the honeycombs. The molecule grafting chemistry is that developed for amino acids.

There can be mentioned, by way of example, grafting techniques involving:
amines with lysine as the typical amino acid; by way of example, primary amines such as lysine can be mentioned to carry out acylation reactions and thus graft all compounds including an acid chloride because the nitrogen of the primary amine is nucleophilic and can react with electrophilic sites,
carboxylic acids with aspartic and glutamic acids as typical amino acids; by way of example, esterification reactions with compounds having alcohol functions can be chosen,
thiols with cysteine as a typical amino acid; by way of example, thiols can be mentioned for grafting compounds including maleimides and/or for creating disulphide bonds with compounds also including thiols and/or for oxidation, alkylation and metalation reactions,
histidines for metal grafting.

This grafting platform can be used in the photovoltaic field or for the detection of molecules. It is possible to contemplate detection of single molecules. It is possible to graft molecules that act, for example, as catalysts. Immobilisation of catalysts increases their stability. It is thus possible to manufacture bio-hybrid materials, for example, for photo-catalysis.

This confinement is also particularly interesting for example for artificial photosynthesis or for carrying out redox reactions.

Particularly advantageously, the specific grafting of metals (or metal ions which are subsequently reduced) into the wells could be used to design metal nanopillars in the cells of the protein structure (wells), for example for nanoelectronics. Their lengths and diameters depend on the protein architecture. The nanopillars are oriented perpendicular to the substrate surface and are regularly spaced. For example, gold nanopillars can be made via a thiol function that binds to the cysteines of the protein's amino acid sequence. For binding metal salts, for example, histidines will be chosen.

The method for manufacturing the nanopillars advantageously comprises the following successive steps of:
functionalising the part of the amino acid sequence of the protein intended to line the wells of the honeycomb, by introducing a metal-reactive amino acid, for example a cysteine amino acid,
crystallising the three-dimensional protein structure on a substrate,
adding a metal, for example in the form of a metal nanoparticle, the metal binding to the metal-reactive amino acid and acting as a metal seed for growing the nanopillar,
growing the metal nanopillar in the well by agglomerating metals around the metal seed.

An alternative way to grow nanopillars in the honeycomb lumen is to replace the C-terminal part of the protein with an amino acid sequence that allows both attraction of the metal salts and reduction in situ without the need to add a reducer. For example, one of the amino acid sequences listed in the following table can be used:

| | |
|---|---|
| Primary amino acid sequence of the protein | MKHHHHHHPMSDYDIPTTENLYFQGAMARK |
| (SEQ ID NO: 15 in the appended sequence | ELSSLEELFRHYGVRYMTLT |
| listing) | KMVEMGFTVNTLVNMTEQELDDVIRTLVDIYR |
| | VDLLVGEKYGIKSAVRAEKR |
| | RLDELERKKLDLFVDVDGKRKADENALDTLSQ |
| Sequence of a mutant with a self-reducing | MKHHHHHHPMSDYDIPTTENLYFQGAMARK |
| sequence at the C-terminal part (SEQ ID NO: | ELSSLEELFRHYGVRYMTLTKMVEMGFTVNTLV |
| 28 in the appended sequence listing): the C- | NMTEQELDDVIRTLVDIYRVDLLVGEKYGIKSAV |
| terminal part has been modified | RAEKRRLDELERKKGGSTGTSVLIATPGV |
| Sequence of a mutant with a self-reducing | MKHHHHHHPGAMARKELSSLEELFRHYGVRY |
| sequence at the C-terminal part (SEQ ID NO: | MTLTKMVEMGFTVNTLVNMTEQELDDVIRTLV |
| 29 in the appended sequence listing): the C- | DIYRVDLLVGEKYGIKSAVRAEKRRLDELERKKG |
| terminal part and the histidine tag have | GSTGTSVLIATPGV |
| been modified | |
| Sequence of a mutant with a self-reducing | MKHHHHHHPMSDYDIPTTENLYFQGAMARK |
| sequence at the C-terminal part (SEQ ID NO: | ELSSLEELFRHYGVRYMTLTKMVEMGFTVNTLV |
| 30 in the appended sequence listing): the C- | NMTEQELDDVIRTLVDIYRVDLLVGEKYGIKSAV |
| terminal part has been modified | RAEKRRLDELERKKGGSWAGAKRLVLRRE |
| Sequence of a mutant with a self-reducing | MKHHHHHHPGAMARKELSSLEELFRHYGVRY |
| sequence at the C-terminal part (SEQ ID NO: | MTLTKMVEMGFTVNTLVNMTEQELDDVIRTLV |
| 31 in the appended sequence listing): the C- | DIYRVDLLVGEKYGIKSAVRAEKRRLDELERKKG |
| terminal part and the histidine tag have | GSWAGAKRLVLRRE |
| been modified | |

These proteins self-assemble in honeycombs perpendicular to the surface. The peptides GGSTGTSVLIATPGV (SEQ ID NO: 32 in the appended sequence listing) and GGSWAGAKRLVLRRE (SEQ ID NO: 33 in the appended sequence listing), coupled to the protein, make it possible to both attract and reduce metal salts.

According to this embodiment, a step could be subsequently carried out during which the protein structure is removed to leave only the nanopillars on the surface of the substrate. This step can be performed, depending on the nature of the substrate, by plasma, or by thermal annealing at a temperature higher than the decomposition temperature of the protein (typically at a temperature higher than 100° C.).

Illustrative and Non-Limiting Examples of One Embodiment:

In this example, the self-assembled protein architecture is obtained by performing the following successive steps of:

1) selecting and synthesising a sequence capable of oligomerising with head-tail interactions to ensure 3D growth of the self-assembly; the head-tail interactions leading to the formation of a primary helix having a groove with sufficient width to allow interlocking of the helices (as shown in FIG. 1C),

2) adding a histidine tag to this sequence at the N-terminal so as to position the self-assembly with a growth perpendicular to the surface.

3) using this construct or so-called mutant sequences to specifically functionalise the honeycomb lumen.

4) producing the protein of interest in bacteria.

5) purifying the protein of interest from the soluble extract of the bacteria that produced it, by performing the following steps of:
collecting the bacteria by centrifugation, breaking their membrane by sonication and then separating the whole of the soluble proteins of the bacteria (containing the protein of interest) from the other elements contained in the bacteria by centrifugation,
purifying the whole:
   in the case of constructs with a histidine tag, purifying the whole by affinity on a nickel-containing resin (for example Nickel-Sepharose type), so as to selectively retain the protein of interest,
   in the case of constructs with a positively charged, negatively charged or hydrophobic tag (in a pH zone between 6 and 9), purifying the assembly by anion exchange (for example on Q-Sepharose type resin), cation exchange (for example on S-Sepharose type resin) or hydrophobic interaction (for example on Phenyl-Sepharose type resin) chromatography techniques respectively,
   in the case of strong receptor/ligand type interactions, purifying the whole by affinity chromatography techniques (for example on a resin containing streptavidin or Strep-Tactin (marketed by IBA Lifesciences) for a construct containing a Strep tag),
optionally performing a second purification on a permeation gel (for example of the Superdex 200 type, marketed by GE Healthcare) in 20 mM Tris-HCl buffer (pH between 7 and 9.0)
concentrating the pure protein to between 0.5 and 5 mg/mL; the protein can be frozen in liquid nitrogen and then stored at −80° C. before use.

6) a volume of x μL of the protein of interest solution (with x ranging, for example, from 5 to 50 μL) is mixed with the same volume of a crystallisation solution so as to form a drop of protein which is then contacted with a surface to be treated. The crystallisation solution may comprise Tris-HCl with a pH of 7 to 9 and ammonium sulphate at a concentration ranging from 40 to 350 mM. The surface to be treated can be made hydrophilic, for example by plasma treatment (“glow-discharge”). A crystallisation tank with a volume ranging, for example, from 500 μL to 5 mL is filled with the crystallisation solution. Then the crystallisation chamber is sealed to initiate vapour diffusion of the drop into the tank. After a time ranging from 4 h to 48 h at room temperature, the surface treated is removed from the crystallisation chamber.

At the end of the method, the resulting material can be viewed by transmission electron microscopy (STEM, TEM) when the support is transparent to electrons. For this, the support is deposited onto an aqueous solution containing a stain (for example 2% uranyl acetate) for 2 minutes. Uranyl acetate will more specifically label the lumen of the honeycombs by interaction of the metal with negative amino acids (Glutamate and Aspartate) that line the lumen of the wells.

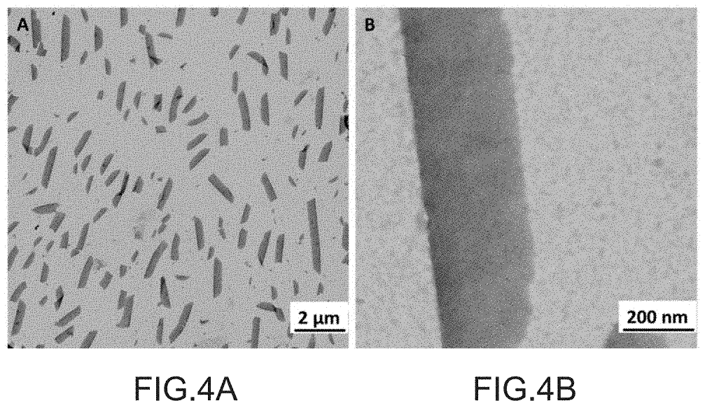

FIGS. 4A and 4B are electron microscope (STEM) pictures of a self-assembled protein structure on a 7 mm² carbon substrate. The honeycomb structure perpendicular to the substrate surface is clearly visible. The coverage rate is 40%.

What is claimed is:

1.-10. (canceled)

11. A method for self-assembling a protein into a three-dimensional honeycomb structure comprising the following sequential steps of:
providing a solution comprising a solvent and a protein, the protein comprising an amino acid sequence corresponding to an oligomerisation domain of a LEAFY protein fused to a tag,
contacting the solution with a substrate,
evaporating the solvent to crystallise the protein, the oligomerisation domain crystallising as a primary helix, each primary helix interacting with six other primary helices, whereby a three-dimensional honeycomb protein structure perpendicular to the substrate is obtained, the protein structure being bound to the substrate through the tag.

12. The method according to claim 11, wherein the method includes an additional step during which another protein corresponding to the oligomerisation domain of the LEAFY protein without a tag is added to the honeycomb structure, whereby the height of the honeycomb structure is increased perpendicular to the substrate.

13. The method according to claim 11, wherein the substrate is of a material selected from a metal, a metalloid or a carbonaceous material.

14. The method according to claim 11, wherein the oligomerisation domain of the LEAFY protein is the oligomerisation domain of Ginkgo biloba.

15. The method according to claim 11, wherein the pitch between each primary helix is less than 10 nm.

16. The method according to claim 11, wherein the internal diameter of a primary helix ranges from 4 nm to 6 nm.

17. The method according to claim 11, wherein the substrate is porous.

18. An assembly comprising a substrate covered with a protein self-assembly in a three-dimensional honeycomb structure,
the protein comprising an amino acid sequence corresponding to an oligomerisation domain of a LEAFY protein, fused to a tag,
the oligomerisation domain being crystallised as a primary helix, each primary helix interacting with six other primary helices, the structure being bound perpendicular to the substrate through the tag.

19. The assembly according to claim 18, wherein the pitch between each primary helix is less than 10 nm.

20. The assembly according to claim 18, wherein the internal diameter of a primary helix ranges from 4 nm to 6 nm.

21. The assembly according to claim 18, wherein the self-assembly covers an area of at least 50 μm².

22. The assembly according to claim 18, wherein the substrate is porous.

23. The assembly according to claim 18, wherein the protein is metallised or functionalised with a metal, a metal salt, an inorganic complex or an organic molecule.

24. A use of an assembly as defined in claim 18, as a photolithography mask, as a signal amplifier of an optical sensor, as a membrane, or as a catalyst.

25. A method for manufacturing nanopillars comprising the following successive steps of:
providing an assembly comprising a substrate covered with a protein self-assembly in a three-dimensional honeycomb structure,
the protein comprising an amino acid sequence corresponding to an oligomerisation domain of a LEAFY protein fused to a tag, and specific amino acids, capable of being functionalised with a metal or a metal salt, lining the honeycomb cells, the oligomerisation domain being crystallised as a primary helix, each primary helix interacting with six other primary helices, the structure being bound perpendicular to the substrate through the tag,
grafting a metal, an inorganic complex or a metal salt onto the specific amino acids, and reducing the metal salt, to form metal nanopillars in the honeycomb cells.

26. The method according to claim 25, wherein the oligomerisation domain of the LEAFY protein is the oligomerisation domain of Ginkgo biloba.
